(19) United States
(12) Patent Application Publication
(10) Pub. No.: US 2002/0147218 A1
(43) Pub. Date: Oct. 10, 2002

(54) Ethanolates of sodium-hydrogen exchanger type-1 inhibitor

(76) Inventors: Timothy Norris, Gales Ferry, CT (US); Rodney M. Weekly, Oakdale, CT (US)

Correspondence Address: Gregg C. Benson, Pfizer Inc., Patent Department, MS 4159, Eastern Point Road, Groton, CT, 06340, US

(21) Appl. No.: 10/060,601

(22) Filed: Jan. 30, 2002

Related U.S. Application Data
(60) Provisional application No. 60/265,456, filed on Jan. 31, 2001.

(51) Int. Cl. A61K 31/4709; C 07D 4 1/02

(52) U.S. Cl. 514/314; 546/167

(57) ABSTRACT

The invention relates to ethanolates of the NHE-1 inhibitor, N-(5-cyclopropyl-1-quinolin-5-yl-1H-pyrazole-4-carbonyl)-guanidine, crystalline forms of the ethanolates, methods of preparing the ethanolates, methods of treatment using the ethanolates and the mesylate salt of the NHE-1 inhibitor prepared using the ethanolates.

### FIELD OF THE INVENTION

This invention relates to novel ethanolates of the sodium-hydrogen exchanger type 1 (NHE-1) inhibitor, N-(5-cyclopropyl-1-quinolin-5-yl-1H-pyrazole-4-carbonyl)-guanidine, novel crystalline forms of the ethanolates, methods of preparing the ethanolates, methods of treatment using the ethanolates and the mesylate salt of the NHE-1 inhibitor prepared using the ethanolates.

### BACKGROUND OF THE INVENTION

The sodium-hydrogen exchanger type 1 (NHE-1) inhibitor, N-(5-cyclopropyl-1-quinolin-5-yl-1 H-pyrazole-4-carbonyl)-guanidine, is useful for the prevention and treatment of myocardial ischemic injury. Myocardial ischemic injury can occur in out-patient as well as in perioperative settings and can lead to the development of sudden death, myocardial infarction or congestive heart failure. It is anticipated that therapies using N-(5-cyclopropyl-1-quinolin-5-yl-1H-pyrazole-4-carbonyl)-guanidine will be life-saving and reduce hospitalizations, enhance quality of life and reduce overall health care costs of high risk patients.

Commonly assigned PCT application published as WO99/43663A1, the disclosure of which is hereby incorporated by reference, discloses a variety of NHE-1 inhibitors, including N-(5-cyclopropyl-1-quinolin-5-yl-1H-pyrazole-4-carbonyl)-guanidine.

There is a need and continuing search in this field of art for compounds useful in the treatment of perioperative myocardial ischemia and forms of such compounds.

### SUMMARY OF THE INVENTION

One aspect of this invention is N-(5-cyclopropyl-1-quinolin-5-yl-1H-pyrazole-4-carbonyl)-guanidine as an ethanolate.

An additional aspect of this invention is N-(5-cyclopropyl-1-quinolin-5-yl-1H-pyrazole-4-carbonyl)-guanidine monoethanolate.

Another aspect of this invention is N-(5-cyclopropyl-1-quinolin-5-yl-1H-pyrazole-4-carbonyl)-guandine monoethanolate which is characterized by an x-ray powder diffraction pattern comprising peaks at about 7.07, 8.60, 14.18, 18.93, 21.34 and 28.54, degrees two-theta, and preferably further comprising peaks at about 16.49, 16.92, 20.70, 23.49, 26.00 and 29.04, degrees two-theta.

An additional aspect of this invention is N-(5-cyclopropyl-1-quinolin-5-yl-1H-pyrazole-4-carbonyl)-guanidine hemiethanolate.

A further aspect of this invention is N-(5-cyclopropyl-1-quinolin-5-yl-1H-pyrazole-4-carbonyl)-guandine hemiethanolate which is characterized by an x-ray powder diffraction pattern comprising peaks at about 7.02,16.44, 18.87, 21.25, and 26.32, degrees two-theta, and preferably further comprising peaks at about 8.55, 12.31, 14.11, 16.91, 23.44, 24.88 and 25.22, degrees two-theta.

Another aspect of this invention are methods for preparing N-(5-cyclopropyl-1-quinolin-5-yl-1H-pyrazole-4-carbonyl)-guanidine monoethanolate comprising:

forming a solution comprising N-(5-cyclopropyl-1-quinolin-5-yl-1H-pyrazole-4-carbonyl)-guanidine in ethanol at a concentration that is about the point of saturation of said compound in ethanol; and

crystallizing N-(5-cyclopropyl-1-quinolin-5-yl-1H-pyrazole-4-carbonyl)-guanidine monoethanolate from said solution.

An additional aspect of this invention are methods for preparing N-(5-cyclopropyl-1-quinolin-5-yl-1H-pyrazole-4-carbonyl)-guanidine hemiethanolate comprising:

forming a solution comprising N-(5-cyclopropyl-1-quinolin-5-yl-1H-pyrazole-4-carbonyl)-guanidine dissolved in ethanol at a concentration that is about the point of saturation of said compound in ethanol;

crystallizing N-(5-cyclopropyl-1-quinolin-5-yl-1H-pyrazole-4-carbonyl)-guanidine monoethanolate from said solution; and

subjecting the N-(5-cyclopropyl-1-quinolin-5-yl-1H-pyrazole-4-carbonyl)-guanidine monoethanolate to drying conditions to form N-(5-cyclopropyl-1-quinolin-5-yl-1H-pyrazole-4-carbonyl)-guanidine hemiethanolate.

A further aspect of this invention are methods for preventing or reducing tissue damage resulting from ischemia or hypoxia comprising administering to a mammal in need of such treatment, preferably a human, a therapeutically effective amount of N-(5-cyclopropyl-1-quinolin-5-yl-1H-pyrazole-4-carbonyl)-guanidine monoethanolate or a pharmaceutical composition comprising said compound.

An additional aspect of this invention are methods for preventing or reducing tissue damage resulting from ischemia or hypoxia comprising administering to a mammal in need of such treatment, preferably a human, a therapeutically effective amount of N-(5-cyclopropyl-1-quinolin-5-yl-1H-pyrazole-4-carbonyl)-guanidine monoethanolate, which is characterized by an x-ray powder diffraction pattern comprising peaks at about 7.07, 8.60, 14.18, 18.93, 21.34 and 28.54, degrees two-theta, or a pharmaceutical composition comprising said compound.

Another aspect of this invention are methods for preventing or reducing tissue damage resulting from ischemia or hypoxia comprising administering to a mammal, preferably a human, in need of such treatment a therapeutically effective amount of N-(5-cyclopropyl-1-quinolin-5-yl-1H-pyrazole-4-carbonyl)-guanidine hemiethanolate or a pharmaceutical composition comprising said compound.

A further aspect of this invention are methods for preventing or reducing tissue damage resulting from ischemia or hypoxia comprising administering to a mammal, preferably a human, in need of such treatment a therapeutically effective amount of N-(5-cyclopropyl-1-quinolin-5-yl-1H-pyrazole-4-carbonyl)-guanidine hemiethanolate, which is characterized by an x-ray powder diffraction pattern comprising peaks at about 7.02, 16.44, 18.87, 21.25, and 26.32, degrees two-theta, or a pharmaceutical composition comprising said compound.

An additional aspect of this invention are pharmaceutical compositions comprising N-(5-cyclopropyl-1-quinolin-5-yl-1H-pyrazole-4-carbonyl)-guanidine monoethanolate and a pharmaceutically acceptable vehicle, diluent or carrier.

A further aspect of this invention are pharmaceutical compositions comprising N-(5-cyclopropyl-1-quinolin-5-yl-1H-pyrazole-4-carbonyl)-guanidine monoethanolate, which is characterized by an x-ray powder diffraction pattern comprising peaks at about 7.07, 8.60, 14.18, 18.93, 21.34 and 28.54, degrees two-theta, and a pharmaceutically acceptable vehicle, diluent or carrier.

Another aspect of this invention are pharmaceutical compositions comprising N-(5-cyclopropyl-1-quinolin-5-yl-1 H-pyrazole-4-carbonyl)-guanidine hemiethanolate and a pharmaceutically acceptable vehicle, diluent or carrier.

An additional aspect of this invention are pharmaceutical compositions comprising N-(5-cyclopropyl-1-quinolin-5-yl-1H-pyrazole-4-carbonyl)-guanidine hemiethanolate, which is characterized by an x-ray powder diffraction pattern comprising peaks at about 7.02, 16.44, 18.87, 21.25, and 26.32, degrees two-theta, and a pharmaceutically acceptable vehicle, diluent or carrier.

A further aspect of this invention are methods for preparing N-(5-cyclopropyl-1-quinolin-5-yl-1H-pyrazole-4-carbonyl)-guandine mesylate comprising combining a compound selected from N-(5-cyclopropyl-1-quinolin-5-yl-1H-pyrazole-4-carbonyl)-guanidine monoethanolate and N-(5-1cyclopropyl-1-quinolin-5-yl-1H-pyrazole-4-carbonyl)-guanidine hemiethanolate, in an aprotic solvent, preferably tetrahydrofuran, with methansulfonic acid at a temperature of about 40° C. to about 80° C., preferably about 50° C. to about 60° C.

Another aspect of this invention are methods for preparing a pharmaceutical composition comprising preparing N-(5-cyclopropyl-1-quinolin-5-yl-1H-pyrazole-4-carbonyl)-guandine mesylate according to a method of this invention and combining said N-(5-cyclopropyl-1-quinolin-5-yl-1H-pyrazole-4-carbonyl)-guandine mesylate with a pharmaceutically acceptable vehicle, diluent or carrier.An additional aspect of this invention are methods for preventing or reducing tissue damage resulting from ischemia or hypoxia comprising administering to a mammal, preferably a human, in need of such treatment a therapeutically effective amount of N-(5-cyclopropyl-1-quinolin-5-yl-1H-pyrazole-4-carbonyl)-guandine mesylate prepared by a method of this invention, or a pharmaceutical composition comprising said compound. A further aspect of this invention are pharmaceutical compositions comprising N-(5-cyclopropyl-1-quinolin-5-yl-1H-pyrazole-4-carbonyl)-guandine mesylate prepared by a method of this invention, and a pharmaceutically acceptable vehicle, diluent or carrier.

In a preferred embodiment of the methods for preparing N-(5-cyclopropyl-1-quinolin-5-yl-1H-pyrazole-4-carbonyl)-guandine monoethanolate aspects of this invention, said crystallization is performed by cooling said solution to a temperature sufficient to effect such crystallization, more preferably wherein said cooling is performed gradually over a period of at least two hours. In another preferred embodiment of the methods for preparing N-(5-cyclopropyl-1-quinolin-5-yl-1H-pyrazole-4-carbonyl)-guandine monoethanolate aspects of this invention, said crystallization is performed by removal of ethanol from said solution by evaporation of an amount sufficient to effect such crystallization.

In a preferred embodiment of the methods for preparing N-(5-cyclopropyl-1-quinolin-5-yl-1H-pyrazole-4-carbonyl)-guandine hemiethanolate aspects of this invention, said drying conditions comprise a vacuum. In another preferred embodiment, said drying conditions comprise heat, preferably at a temperature of about 40° C. to about 45° C. In a further preferred embodiment, said drying conditions comprise heat and vacuum, preferably wherein said heat is at a temperature of about 40° C. to about 45° C. and said vacuum is at or less than about 15 mm Hg. In an additional preferred embodiment, the N-(5-cyclopropyl-1-quinolin-5-yl-1 H-pyrazole-4-carbonyl)-guandine monoethanolate is subjected to drying conditions for at least about five hours. In another preferred embodiment, said crystallization is performed by cooling said solution to a temperature sufficient to effect such crystallization. In an additional preferred embodiment, said crystallization is performed by removal of ethanol from said solution in an amount sufficient to effect such crystallization.

The term “hemiethanolate” as used herein means crystalline N-(5-cyclopropyl-1-quinolin-5-yl-1H-pyrazole-4-carbonyl)-guandine having about 0.4 to about 0.6 molecules of ethanol to each molecule of N-(5-cyclopropyl-1-quinolin-5-yl-1H-pyrazole-4-carbonyl)-guandine, within the crystal structure thereof.

The term “monoethanolate” as used herein means crystalline N-(5-cyclopropyl-1-quinolin-5-yl-1H-pyrazole-4-carbonyl)-guandine having about 0.9 to about 1.1 molecules of ethanol to each molecule of N-(5-cyclopropyl-1-quinolin-5-yl-1H-pyrazole-4-carbonyl)-guandine, within the crystal structure thereof.

The term “point of saturation” as used herein means the concentration at which a solution contains a quantity of a dissolved substance such that no more of that substance will dissolve under the existing conditions of such solution (for example, temperature, pH, pressure).

Those skilled in the art will recognize that certain compounds of this invention will contain one or more atoms that may be in a particular stereochemical or geometric configuration, giving rise to stereoisomers and configurational isomers. All such isomers and mixtures thereof are included in this invention. Those skilled in the art will recongnized that N-(5-cyclopropyl-1-quinolin-5-yl-1H-pyrazole-4-carbonyl)-guanidine monoethanolate, N-(5-cyclopropyl-1-quinolin-5-yl-1H-pyrazole-4-carbonyl)-guanidine hemiethanolate and N-(5-cyclopropyl-1-quinolin-5-yl-1 H-pyrazole-4-carbonyl)-guanidine mesylate can exist in several tautomeric forms. All such tautomeric forms are considered as part of this invention.

### BRIEF DESCRIPTION OF THE DRAWINGS

FIG. 1 is a characteristic x-ray powder diffraction spectrum of the N-(5-cyclopropyl-1-quinolin-5-yl-1H-pyrazole-4-carbonyl)-guanidine monoethanolate crystalline form of this invention prepared by the method of Example 5.

FIG. 2 is a characteristic x-ray powder diffraction spectrum of the N-(5-cyclopropyl-1-quinolin-5-yl-1H-pyrazole-4-carbonyl)-guanidine hemiethanolate crystalline form of this invention prepared by the method of Example 6.

### DETAILED DESCRIPTION OF THE INVENTION

Scheme I illustrates the process of preparing N-(5-cyclopropyl-1-quinolin-5-yl-1H-pyrazole-4-carbonyl)-guanidine ethanolate. This and other processes are exemplified in the Experimental Procedures section.

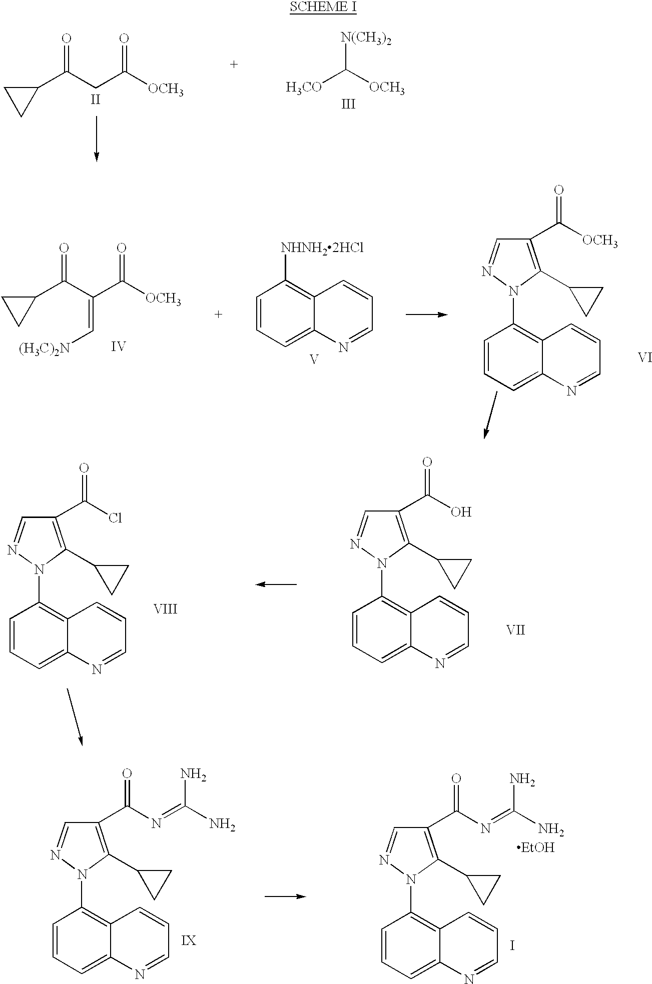

As illustrated in Scheme I, the Formula II compound is combined with the Formula III compound to prepare the Formula IV compound. The Formula IV compound is cyclized with the Formula V compound to form the Formula VI ester. The Formula VI ester is hydrolyzed to prepare the Formula VII acid. The Formula VII acid is treated with thionyl chloride to form the Formula VIII activated acid chloride. The Formula VII compound is then coupled with guanidine to form the Formula IX NHE-1 inhibitor, N-(5-cyclopropyl-1-quinolin-5-yl-1H-pyrazole-4-carbonyl)-guanidine.

The Formula I monoethanolate of N-(5-cyclopropyl-1-quinolin-5-yl-1H-pyrazole-4-carbonyl)-guanidine may be prepared by dissolving the NHE-1 inhibitor in ethanol, followed by crystallization of the compound from the solution. The compound that crystallizes from the ethanol solution is N-(5-cyclopropyl-1-quinolin-5-yl-1H-pyrazole-4-carbonyl)-guanidine monoethanolate.

Crystallization may be achieved by methods well known by those skilled in the art. For example, the monoethanolate may be crystallized by sufficiently cooling the ethanol solution to cause the compound to crystallize out of solution.

The monoethanolate may also be crystallized from the ethanol solution by evaporation of sufficient ethanol so as to cause the compound to crystallize out of solution. Evaporation of ethanol may be performed by heating the ethanol solution, preferably to a temperature sufficient to cause the solution to boil. Alternatively, a vacuum or a combination of vacuum and heat may be employed to effect such evaporation.

It is preferable that the initial concentration of N-(5-cyclopropyl-1-quinolin-5-yl-1H-pyrazole-4-carbonyl)-guanidine in the solution be near the point of saturation. It will be apparent to those skilled in the art that the point of saturation will be dependent on the temperature of the solution and may be dependent on other factors such as pH of the solution, extraneous substances in the solution and/or the barometric pressure.

It will also be apparent to those skilled in the art that regardless of the initial concentration of N-(5-cyclopropyl-1-quinolin-5-yl-1H-pyrazole-4-carbonyl)-guanidine in the ethanol solution, the method used for crystallization of the monoethanolate from the ethanol solution will cause the formation of a mixture having a concentration of N-(5-cyclopropyl-1-quinolin-5-yl-1H-pyrazole-4-carbonyl)-guanidine that is at or near the point of saturation of said compound in ethanol. For example, if the method of crystallization that is employed is cooling of the solution, the saturation point will be approached as the solution is cooled. If the method of crystallization is evaporation of ethanol from the solution, the saturation point will be approached as ethanol is removed from the solution.

In a preferred embodiment, N-(5-cyclopropyl-1-quinolin-5-yl-1H-pyrazole-4-carbonyl)-guanidine is dissolved in boiling ethanol to form a solution and the monoethanolate is crystallized from such solution. It will be apparent to those skilled in the art that the temperature at which a solution of ethanol and N-(5-cyclopropyl-1-quinolin-5-yl-l H-pyrazole-4-carbonyl)-guanidine boils may be dependent on factors such as barometric pressure and the concentration of N-(5-cyclopropyl-1-quinolin-5-yl-1H-pyrazole-4-carbonyl)-guanidine in such solution.

In another preferred embodiment, the monoethanolate is crystallized from the ethanol solution by gradually cooling the solution, preferably to room temperature, and preferably over a period of at least about one-half hour, more preferably over a period of at least about one hour, even more preferably over a period of at least about two hours and still even more preferably over a period of at least about four hours.

Large crystals of the ethanolate may be prepared by dissolving N-(5-cyclopropyl-1-quinolin-5-yl-1H-pyrazole-4-carbonyl)-guanidine in boiling ethanol followed by gradual cooling of the solution, as described above, to afford large white crystals of the monoethanolate. Single crystal x-ray analysis of a crystal prepared by this method confirms the monoethanolate, showing a one to one relationship between the N-(5-cyclopropyl-1-quinolin-5-yl-1H-pyrazole-4-carbonyl)-guanidine molecule and the ethanole molecule. However, the position of the ethanol within the structure is found to be disordered.

In a further preferred embodiment for preparing the monoethanolate, wherein N-(5-cyclopropyl-1-quinolin-5-yl-1H-pyrazole-4-carbonyl)-guanidine has been prepared by combining guanidine with a suspension or solution of 5-cyclopropyl-1-quinolin-5-yl-1H-pyrazole-4-carbonyl chloride in a relatively inert reaction solvent such as tetrahydrofuran, to form a solution of N-(5-cyclopropyl-1-quinolin-5-yl-1H-pyrazole-4-carbonyl)-guanidine in the reaction solvent, the monoethanolate may be prepared by evaporation of the reaction solvent, such as by distillation of the solvent solution, followed by the addition of ethanol and further evaporation of the solution, to afford the monoethanolate. Multiple cycles of ethanol addition and evaporation may be employed to ensure substantially complete removal of the tetrahydrofuran prior to final crystallization into the monoethanolate.

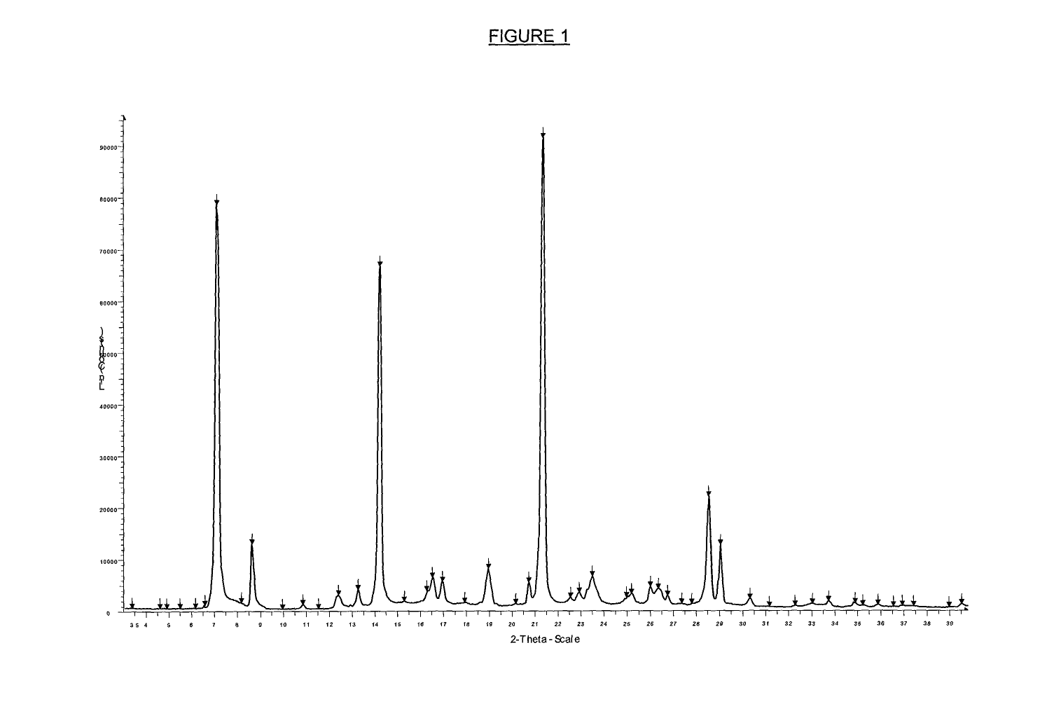

N-(5-Cyclopropyl-1-quinolin-5-yl-1H-pyrazole-4-carbonyl)-guanidine monoethanolate prepared by the preceding method results in a crystal form that is characterized by a powder x-ray diffraction pattern as substantially represented by FIG. 1. The principal peaks observed are at about 7.07, 8.60, 14.18, 18.93, 21.34 and 28.54, degrees 2 theta. Additional principal peaks observed are at about 16.49, 16.92, 20.70, 23.49, 26.00 and 29.04, degrees 2 theta.

The hemiethanolate of N-(5-cyclopropyl-1-quinolin-5-yl-1H-pyrazole-4-carbonyl)-guanidine may be prepared by subjecting the monoethanolate to drying conditions such as heat and/or vacuum. It should be noted that drying of the monoethanolate at room temperature under a stream of nitrogen gas is not sufficient to appreciably convert the monoethanolate to the hemiethanolate. Therefore, the drying conditions should be sufficiently robust for the conversion to occur.

In a preferred embodiment for preparing the hemiethanolate, N-(5-cyclopropyl-1-quinolin-5-yl-1H-pyrazole-4-carbonyl)-guanidine monoethanolate is placed in a vacuum oven at a temperature of about 40° C. to about 45° C., at a barometric pressure of about 15 mm Hg, preferably for at least about two hours, more preferably at least about five hours and even more preferably at least about 10 hours, to afford N-(5-cyclopropyl-1-quinolin-5-yl-1H-pyrazole-4-carbonyl)-guanidine hemiethanolate.

In another further preferred embodiment for preparing the hemiethanolate, N-(5-cyclopropyl-1-quinolin-5-yl-1H-pyrazole-4-carbonyl)-guanidine monoethanolate is prepared by combining guanidine with 5-cyclopropyl-1-quinolin-5-yl-1H-pyrazole-4-carbonyl chloride that is suspended in tetrahydrofuran, to form a solution of N-(5-cyclopropyl-1-quinolin-5-yl-1H-pyrazole-4-carbonyl)-guanidine in tetrahydrofuran. The solution is distilled to remove the tetrahydrofuran leaving a residue. The residue is dissolved in ethanol and the ethanol is distilled. Multiple cycles of ethanol addition and distillation are employed to ensure substantially complete removal of the tetrahydrofuran prior to final crystallization into the monoethanolate. The monoethanolate is then placed in a vacuum oven at a temperature of about 40° C. to about 45° C., at a barometric pressure of about 15 mm Hg, preferably for at least about two hours, more preferably at least about five hours and even more preferably at least about 10 hours, to afford N-(5-cyclopropyl-1-quinolin-5-yl-1H-pyrazole-4-carbonyl)-guanidine hemiethanolate.

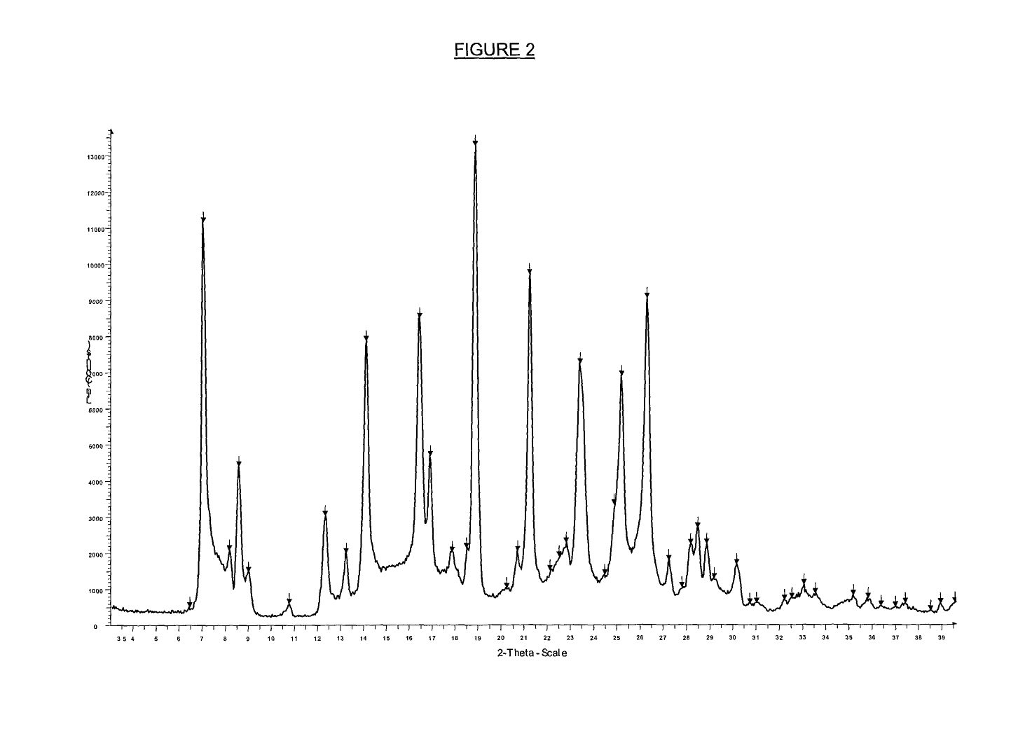

N-(5-Cyclopropyl-1-quinolin-5-yl-1H-pyrazole-4-carbonyl)-guanidine hemiethanolate prepared by the immediately preceding method results in a crystal form that is characterized by a powder x-ray diffraction pattern as substantially represented by FIG. 2. The principal peaks observed are at about 7.02, 16.44, 18.87, 21.25, and 26.32, degrees 2 theta. Additional principal peaks observed are at about 8.55, 12.31, 14.11, 16.91, 23.44, 24.88 and 25.22, degrees 2 theta.

N-(5-Cyclopropyl-1-quinolin-5-yl-1H-pyrazole-4-carbonyl)-guanidine mesylate may be prepared by reacting the N-(5-cyclopropyl-1-quinolin-5-yl-1H-pyrazole-4-carbonyl)-guanidine monoethanolate with methansulfonic acid. The mesylate salt may also be prepared by reacting methansulfonic acid with N-(5-cyclopropyl-1-quinolin-5-yl-1H-pyrazole-4-carbonyl)-guanidine hemiethanolate. Preferably the monoethanolate or hemiethanolate, as applicable, is reacted with methansulfonic acid in an aprotic solvent such as tetrahydrofuran or a mixture of about 60% to about 90% acetone and the remainder 1-methyl-2-pyrrolidinone at a temperature of about 40° C. to about 80° C.

The starting material and reagents for the above described compounds are readily available or can be easily synthesized by those skilled in the art using conventional methods of organic synthesis.

Administration of the compounds prepared by a method of this invention may be achieved by any method which delivers a compound of this invention preferentially to the desired tissue (e.g., liver and/or cardiac tissues). These methods include oral routes, parenteral, intraduodenal routes, etc. Generally, the compounds of the present invention are administered in single (e.g., once daily) or multiple doses or via constant infusion.

The ethanolates and the mesylate salt of N-(5-cyclopropyl-1-quinolin-5-yl-1H-pyrazole-4-carbonyl)-guanidine prepared by a method of this invention are useful, for example, in preventing, reducing or minimizing damage effected directly to any tissue that may be susceptible to ischemia/ reperfusion injury (e.g., heart, brain, lung, kidney, liver, gut, skeletal muscle, retina) as the result of an ischemic event (e.g., myocardial infarction). The compound is therefore usefully employed prophylactically to prevent, blunt or stem tissue damage (e.g., myocardial tissue) in patients who are at risk for ischemia (e.g., myocardial ischemia).

Generally, the compounds prepared by a method of this invention are administered orally, or parenterally (e.g., intravenous, intramuscular, subcutaneous or intramedullary). Topical administration may also be indicated, for example, where the patient is suffering from gastrointestinal disorders or whenever the medication is best applied to the surface of a tissue or organ as determined by the attending physician.

The amount and timing of compound administered will, of course, be dependent on the subject being treated, on the severity of the affliction, on the manner of administration and on the judgement of the prescribing physician. Thus, because of patient to patient variability, the dosages given below are a guideline and the physician may titrate doses of the drug to achieve the treatment that the physician considers appropriate for the patient. In considering the degree of treatment desired, the physician must balance a variety of factors such as age of the patient, presence of preexisting disease, as well as presence of other diseases (e.g., cardiovascular disease).

For example, in one mode of administration, a compound of this invention may be administered just prior to surgery (e.g., within twenty-four hours before surgery for example cardiac surgery) during or subsequent to surgery (e.g., within twenty-four hours after surgery) where there is risk of myocardial ischemia. The compound may also be administered in a chronic daily mode.

An amount of a compound of this invention is used that is effective for ischemic protection. A preferred dosage is about 0.001 to 100 mg/kg/day of the compound. An especially preferred dosage is about 0.01 to 50 mg/kg/day of the compound.

The compounds of the present invention are generally administered in the form of a pharmaceutical composition comprising at least one of the compounds of this invention together with a pharmaceutically acceptable vehicle, carrier or diluent. Thus, the compounds of this invention can be administered individually or together in any conventional oral, parenteral, rectal or transdermal dosage form.

For oral administration a pharmaceutical composition can take the form of solutions, suspensions, tablets, pills, capsules, powders, and the like. Tablets containing various excipients such as sodium citrate, calcium carbonate and calcium phosphate are employed along with various disintegrants such as starch and preferably potato or tapioca starch and certain complex silicates, together with binding agents such as polyvinylpyrrolidone, sucrose, gelatin and acacia. Additionally, lubricating agents such as magnesium stearate, sodium lauryl sulfate and talc are often very useful for tabletting purposes. Solid compositions of a similar type are also employed as fillers in soft and hard-filled gelatin capsules; preferred materials in this connection also include lactose or milk sugar as well as high molecular weight polyethylene glycols. When aqueous suspensions and/or elixirs are desired for oral administration, the compounds of this invention can be combined with various sweetening agents, flavoring agents, coloring agents, emulsifying agents and/or suspending agents, as well as such diluents as water, ethanol, propylene glycol, glycerin and various like combinations thereof.

For purposes of parenteral administration, solutions, for example, in sesame or peanut oil or in aqueous propylene glycol can be employed, as well as sterile aqueous solutions of the corresponding water-soluble salts. Such aqueous solutions may be suitably buffered, if necessary, and the liquid diluent first rendered isotonic with sufficient saline or glucose. These aqueous solutions are especially suitable for intravenous, intramuscular, subcutaneous and intraperitoneal injection purposes. In this connection, the sterile aqueous media employed are all readily obtainable by standard techniques well-known to those skilled in the art.

For purposes of transdermal (e.g.,topical) administration, dilute sterile, aqueous or partially aqueous solutions (usually in about 0.1% to 5% concentration), otherwise similar to the above parenteral solutions, are prepared.

Methods of preparing various pharmaceutical compositions with a certain amount of active ingredient are known, or will be apparent in light of his disclosure, to those skilled in this art. For examples of methods of preparing pharmaceutical compositions, see Remington: The Science and Practice of Pharmacy, Mack Publishing Company, Easton, Pa., 19th Edition 1995.

Pharmaceutical compositions according to the invention may contain for example 0.0001%-95% of a compound of this invention. In any event, the composition or formulation to be administered will contain a quantity of the compound in an amount effective to treat the disease/condition of the subject being treated.

The compounds of this invention generally will be administered in a convenient formulation. The following formulation examples are illustrative only and are not intended to limit the scope of the present invention.

In the formulations which follow, “active ingredient” means either the monoethanolate or the hemiethanolate of N-(5-cyclopropyl-1-quinolin-5-yl-1H-pyrazole-4-carbonyl)-guanidine of this invention or the mesylate of N-(5-cyclopropyl-1-quinolin-5-yl-1H-pyrazole-4-carbonyl)-guanidine of this invention. Active ingredient above may also be a combination of two compounds such compounds or all three such compounds.

Formulation 1: Gelatin Capsules
Hard gelatin capsules are prepared using the following:

| Ingredient | Quantity (mg/capsule) |
|---|---|
| Active ingredient | 0.25-100 |
| Starch, NF | 0-650 |
| Starch flowable powder | 0-50 |
| Silicone fluid 350 centistokes | 0-15 |

A tablet formulation is prepared using the ingredients below:

Formulation 2: Tablets

| Ingredient | Quantity (mg/tablet) |
|---|---|
| Active ingredient | 0.25-100 |
| Cellulose, microcrystalline | 200-650 |
| Silicon dioxide, fumed | 10-650 |
| Stearate acid | 5-15 |

The components are blended and compressed to form tablets.

Alternatively, tablets each containing 0.25-100 mg of active ingredients are made up as follows:

Formulation 3: Tablets

| Ingredient | Quantity (mg/tablet) |
|---|---|
| Active ingredient | 0.25-100 |
| Starch | 45 |
| Cellulose, microcrystalline | 35 |
| Polyvinylpyrrolidone (as 10% solution in | 4 |
| water) | |
| Sodium carboxymethyl cellulose | 4.5 |
| Magnesium stearate | 0.5 |
| Talc | 1 |

The active ingredient, starch, and cellulose are passed through a No. 45 mesh U.S. sieve and mixed thoroughly. The solution of polyvinylpyrrolidone is mixed with the resultant powders which are then passed through a No. 14 mesh U.S. sieve. The granules so produced are dried at 50°-60° C. and passed through a No. 18 mesh U.S. sieve. The sodium carboxymethyl starch, magnesium stearate, and talc, previously passed through a No. 60 U.S. sieve, are then added to the granules which, after mixing, are compressed on a tablet machine to yield tablets.

Suspensions each containing 0.25-100 mg of active ingredient per 5 ml dose are made as follows:

Formulation 4: Suspensions

| | |
|---|---|
| Ingredient | Quantity (mg/5 ml) |

| | | |
|---|---|---|
| Active ingredient | 0.25-100 | mg |
| Sodium carboxymethyl cellulose | 50 | mg |
| Syrup | 1.25 | mg |
| Benzoic acid solution | 0.10 | mL |

| | |
|---|---|
| Flavor | q.v. |
| Color | q.v. |

| | | |
|---|---|---|
| Purified Water to | 5 | mL |

The active ingredient is passed through a No. 45 mesh U.S. sieve and mixed with the sodium carboxymethyl cellulose and syrup to form a smooth paste. The benzoic acid solution, flavor, and color are diluted with some of the water and added, with stirring. Sufficient water is then added to produce the required volume. An aerosol solution is prepared containing the following ingredients:

Formulation 5: Aerosol

| | Quantity (% by |
|---|---|
| Ingredient | weight) |
| Active ingredient | 0.25 |
| Ethanol | 25.75 |
| Propellant 22 (Chlorodifluoromethane) | 74.00 |

The active ingredient is mixed with ethanol and the mixture added to a portion of the propellant 22, cooled to 30° C., and transferred to a filling device. The required amount is then fed to a stainless steel container and diluted with the remaining propellant. The valve units are then fitted to the container. Suppositories are prepared as follows:

Formulation 6: Suppositories

| | Quantity (mg/ |
|---|---|
| Ingredient | suppository) |
| Active ingredient | 250 |
| Saturated fatty acid glycerides | 2,000 |

The active ingredient is passed through a No. 60 mesh U.S. sieve and suspended in the saturated fatty acid glycerides previously melted using the minimal necessary heat. The mixture is then poured into a suppository mold of nominal 2 g capacity and allowed to cool.

An intravenous formulation is prepared as follows:

Formulation 7: Intravenous Solution

| | |
|---|---|
| Ingredient | Quantity |

| | | |
|---|---|---|
| Active ingredient | 25 | mg |
| Isotonic saline | 1,000 | mL |

The solution of the above ingredients is intravenously administered to a patient.

### Experimental Procedures

NMR spectra were recorded on a Varian XL-300 (Varian Co., Palo Alto, Calif.), a Bruker AM-300 spectrometer (Bruker Co., Billerica, Mass.) or a Varian Unity 400 at about 23° C. at 300 or 400 MHz for proton. DMSO was the solvent. The peak shapes are denoted as follows: s=singlet; d=doublet; t=triplet, q=quartet; m=multiplet; bs=broad singlet.

Powder x-ray diffraction analysis was performed on a Siemens D5000 powder X-ray diffractometer (Bruker AXS, Inc., Madison, Wis., formerly Siemens) equipped with copper radiation and using theta/2 theta geometry and a Kevex solid state detector (Thermo Noran, Middleton, Wis.).

Single crystal x-ray analysis was performed at room temperature using a using a Siemens P4 diffractometer. Atomic scattering factors were based upon the International Tables for X-Ray Crystallography, Vol. IV, pp. 55, 99, 149 Birmingham: Kynoch Press, 1974. Crystal structure plotting was carried out using SHELXTL™ software (version 5.1, Bruker AXS, Inc., Madison, Wis.).

### EXAMPLE 1

### Methyl-3-Cyclopropyl-2-Dimethylenamino-3-Oxopropanoate.

Methyl-3-cyclopropyl-3-oxopropanoate (15 g) and N, N-dimethylformamide dimethylacetal (14.7 mL) were heated at 75° C. for 1.5 hours under nitrogen atmosphere. The resulting orange oil was then cooled to room temperature affording crude methyl-3-cyclopropyl-2-dimethylenamino-3-oxopropanoate.

Thin-layer chromatography (TLC) analysis (1:1 EtOAc/hexanes) indicated disappearance of starting material and appearance of a minor less polar spot and a major more polar spot (methyl-3-cyclopropyl-2-dimethylenamino-3-oxopropanoate).

### EXAMPLE 2

### 5-Cyclopropyl-1-Quinolin-5-yl-1H-Pyrazole-4-Carboxlic Acid Methyl Ester.

Crude methyl-3-cyclopropyl-2-dimethylenamino-3-oxopropanoate (20.9 g) prepared by the method of Example 1 was diluted with ethanol (250 mL). Triethylamine (34.4 mL) was added followed by quinolin-5-yl-hydrazine (22.9 g). Slight gas evolution was observed following the addition of quinolin-5-yl-hydrazine. The resulting heterogeneous mixture was heated at reflux (78° C.) under nitrogen atmosphere for 2 hours. The resulting mixture was then cooled to room temperature. TLC analysis (1:1 ethyl acetate/hexanes) indicated a slightly less polar spot (5-cyclopropyl-1-quinolin-5-yl-1H-pyrazole-4-carboxylic acid methyl ester). The reaction mixture was then concentrated by evaporation of excess solvent. Ethyl acetate (300 mL) and 0.1 N hydrochloric acid (400 mL) were added to the residue. The resulting emulsion was stirred for 10 minutes at room temperature and then filtered through a pad of Celite® (Celite Corporation, Lompoc,Calif.) to remove solids. The resulting biphasic mixture was separated. The aqueous layer was extracted with ethyl acetate (2×300 mL). The combined organic layers were washed with 0.1 N hydrochloric acid (2×300 mL), then dried over sodium sulfate, and concentrated by evaporation of the solvent. Hot isopropyl ether (80 mL) was added to the resulting residue. The resulting cloudy solution was stirred for two minutes. Hexanes (125 mL) were then added and the solids that formed were allowed to granulate overnight. The resulting solids were collected by filtration to afford the title compound as a yellow orange powder (20.8 g).

### EXAMPLE 3

### 5-Cyclopropyl-1-Quinolin-5-yl-1H-Pyrazole-4-Carboxylic Acid.

To a solution of 5-cyclopropyl-1-quinolin-5-yl-1H-pyrazole-4-carboxylic acid methyl ester (20 g) in methanol (120 mL) was added 2N sodium hydroxide (54.5 mL). The resulting solution was heated at reflux (65° C.) for 1.5 hour under nitrogen atmosphere, and then allowed to cool to room temperature. TLC analysis (1:1 ethyl acetate/hexanes) indicated disappearance of starting material. The methanol was removed under vacuum with gentle heating (35° C.) on a rotovap. The basic aqueous layer was then washed with ethyl acetate (2×100 mL). The resulting basic aqueous layer was acidified slowly to about pH 1 to 2 with concentrated hydrochloric acid. The product precipitated out during acidification. The slurry was stirred at room temperature for 0.5 h, then the solids were collected by filtration. The solids were washed with 1N hydrochloric acid (2×25 mL) and dried to afford the title compound as a pale brown solid (18.8 g).

### EXAMPLE 4

### 5-Cyclopropyl-1-Quinolin-5-yl-1H-Pyrazole-4-Carbonyl Chloride.

A glass-lined 100 liter reactor under nitrogen atmosphere was charged with 63 liters of toluene and 5.2 kg of 5-cyclopropyl-1-quinolin-5-yl-1H-pyrazole-4-carboxylic acid. The reactor was heated to boiling and 11 liters of distillate were collected to azeotropically dry the system. The vessel was cooled to about 40° C. and 2.4 kg of thionyl chloride were added. The reactor was heated to about 75° C. and this temperature was maintained for about 13 hours. The reactor was cooled to about 20° C. and the solids present were isolated by filtration. The solids were rinsed with toluene affording a “wet cake” of the title compound.

### EXAMPLE 5

### N-(5-Cyclopropyl-1-Quinolin-5-yl-1H-Pyrazole-4-Carbonyl)-Guanidine Monoethanolate.

A 100 liter glass-lined vessel containing 64 liters of tetrahydrofuran (THF) was charged under nitrogen atmosphere with the 5-cyclopropyl-1-quinolin-5-yl-1H-pyrazole-4-carbonyl chloride wet cake from Example 4. Agitation was used to suspend the solids in the vessel.

A separate 200 liter glass-lined reactor under nitrogen atmosphere was charged with 31 liters of water, 5.1 kg of potassium hydroxide pellets and 3.60 kg of guanidine hydrochloride, to reach a pH of 14. Additional potassium hydroxide may be used so long as the pH reaches 14. Sodium hydroxide may be substituted for potassium hydroxide.

The resulting solution was cooled to 0-5° C. The 5-cyclopropyl-1-quinolin-5-yl-1H-pyrazole-4-carbonyl chloride/THF suspension was added to the 200 liter reactor over about 30 minutes while maintaining reactor temperature at about 0° C. to about 5° C. The vessel was warmed to about 20° C. and stirred for 90 minutes. The agitation of the reactor was stopped and two liquid layers formed on standing. The lower aqueous layer was removed and extracted two additional times with 19 liters of THF (38 liters total). The three THF fractions were combined and stirred with activated carbon and filter aid for 1 hour at about 50° C. The activated carbon/filter aid suspension was filtered hot and rinsed with THF.

The resulting filtrate was transferred to a 200 liter vessel, configured for atmospheric distillation under nitrogen. The vessel was heated to boiling and about 100 liters of distillate were collected. Ninety-four liters of ethanol (100%) were charged to the distillation vessel and the distillation was resumed, collecting another 94 liters of distillate. A second charge of ethanol (94 liters) was made to the distillation vessel and the distillation was resumed, collecting another 94 liters of distillate. A third charge of ethanol (94 liters) was made to the distillation vessel and the distillation was resumed, collecting another 82 liters of distillate. N-(5-cyclopropyl-1-quinolin-5-yl-1H-pyrazole-4-carbonyl)-guanidine monoethanolate crystallized near the end of the distillation. The vessel was then cooled to about 20° C. and the solids were isolated by filtration and rinsed with ethanol, affording the title compound.

N-(5-Cyclopropyl-1-quinolin-5-yl-1H-pyrazole-4-carbonyl)-guanidine monoethanolate prepared by the method of this example resulted in a powder x-ray diffraction pattern as substantially represented by FIG. 1.

### EXAMPLE 6

### N-(5-Cyclopropyl-1-Quinolin-5-yl-1H-Pyrazole-4-Carbonyl)-Guanidine Hemiethanolate.

The N-(5-cyclopropyl-1-quinolin-5-yl-1H-pyrazole-4-carbonyl)-guanidine monoethanolate from Example 5 was dried in a vacuum oven at a temperature of about 40° C. to about 45° C. and at a pressure of about 15 mm Hg, affording 5.10 kg of N-(5-cyclopropyl-1-quinolin-5-yl-1H-pyrazole-4-carbonyl)-guanidine hemiethanolate.

The ethanol content of the hemiethanolate is confirmed by NMR analysis.

| δ | Mult | J-Hz | Assign. | Integration |
|---|---|---|---|---|
| 9.0 | dd | 4.1, 1.9 | Ar—H | 1 H |
| 8.22 | d | 8.4 | Ar—H | 1 H |
| 8.01 | s | | Ar—H (pyrazole) | 1 H |
| 7.92 | dd | 8.4, 7.5 | Ar—H | 1 H |
| 7.79 | dd | 7.5, 1.1 | Ar—H | 1 H |
| 7.62 | m | | Ar—H | 2 H |
| 4.39 | t | 5.1 | OH (EtOH) | 0.5 H |
| 3.45 | m | | OH2 (EtOH) | 1 H |
| 1.97 | m | | OH (cyclopropyl) | 1 H |
| 1.07 | t | 6.9 | CH₃ (EtOH) | 1.5 H |
| 0.58 | m | | CH₂—CH₂ (cyclopropyl) | 4 H |

N-(5-Cyclopropyl-1-quinolin-5-yl-1H-pyrazole-4-carbonyl)-guanidine hemiethanolate prepared by the method of this example resulted in a powder x-ray diffraction pattern as substantially represented by FIG. 2.

### EXAMPLE 7

### N-(5-Cycloproryl-1-Quinolin-5-yl-1H-Pyrazole-4-Carbonyl)-Guanidine Mesylate.

A 50 liter glass-lined reactor was charged with 44 liters of THF, 4.4 liters of dimethylsulfoxide and 4.81 kg of N-(5-cyclopropyl-1-quinolin-5-yl-1H-pyrazole-4-carbonyl)-guanidine hemiethanolate. The reactor was warmed to about 35° C. under nitrogen, forming a solution. The solution was filtered into a second vessel to remove trace insoluble material. A solution of 1246 g of methanesulfonic acid in THF (about 8 liters) was prepared in an addition flask over the vessel containing the filtrate. The vessel containing the filtrate was warmed to about 53° C. and the acid solution was added slowly over a 1 hour period. N-(5-Cyclopropyl-1-quinolin-5-yl-1H-pyrazole-4-carbonyl)-guanidine mesylate crystallized near the end of the acid addition. The solids were isolated by filtration, rinsed with THF and vacuum dried, affording 5.13 kg of N-(5-cyclopropyl-1-quinolin-5-yl-1H-pyrazole-4-carbonyl)-guanidine mesylate.

### EXAMPLE 8

### N-(5-Cyclopropyl-1-Quinolin-5-yl-1H-Pyrazole-4-Carbonyl)-Guanidine Monoethanolate-Large Crystals.

One gram of N-(5-cyclopropyl-1-quinolin-5-yl-1H-pyrazole-4-carbonyl)-guanidine was dissolved in 36 ml of boiling ethanol (100%). The solution was then allowed to gradually cool overnight to room temperature resulting in the formation of large white crystals of N-(5-cyclopropyl-1-quinolin-5-yl-1H-pyrazole-4-carbonyl)-guanidine monoethanolate. Yield was 0.95 grams.

The monoethanolate was confirmed by single-crystal x-ray crystallography analysis. Prior to x-ray analysis, the crystal was removed directly from the ethanol mother liquor and sealed in epoxy. The single crystal x-ray analysis demonstrated a monoethanolate crystal having a one to one relationship between the N-(5-cyclopropyl-1-quinolin-5-yl-1H-pyrazole-4-carbonyl)-guanidine molecule and the ethanole molecule. However, the position of the ethanol within the structure was found to be disordered.

The ethanol content of the monoethanolate was also confirmed by NMR analysis using the title compound prepared according to this Example 8. However, after the formation of the crystals, the ethanol was decanted and the solid was dried at room temperature over a stream of nitrogen gas.

Although certain presently preferred embodiments of the invention have been described herein, it will be apparent to those skilled in the art that variations and modifications of the described embodiments may be made without departing from the spirit and scope of the invention.

1. N-(5-cyclopropyl-1-quinolin-5-yl-1H-pyrazole-4-carbonyl)-guanidine as an ethanolate.

2. N-(5-Cyclopropyl-1-quinolin-5-yl-1H-pyrazole-4-carbonyl)-guanidine monoethanolate.

3. N-(5-Cyclopropyl-1-quinolin-5-yl-1H-pyrazole-4-carbonyl)-guanidine monoethanolate of claim 2 which is characterized by an x-ray powder diffraction pattern comprising peaks at about 7.07, 8.60, 14.18, 18.93, 21.34 and 28.54, degrees two-theta.

4. N-(5-Cyclopropyl-1-quinolin-5-yl-1H-pyrazole-4-carbonyl)-guanidine monoethanolate of claim 3 which is characterized by an x-ray powder diffraction pattern further comprising peaks at about 16.49, 16.92, 20.70, 23.49, 26.00 and 29.04, degrees two-theta.

5. N-(5-Cyclopropyl-1-quinolin-5-yl-1H-pyrazole-4-carbonyl)-guanidine hemiethanolate.

6. N-(5-Cyclopropyl-1-quinolin-5-yl-1H-pyrazole-4-carbonyl)-guanidine hemiethanolate of claim 5 which is characterized by an x-ray powder diffraction pattern comprising peaks at about 7.02, 16.44, 18.87, 21.25, and 26.32, degrees two-theta.

7. N-(5-Cyclopropyl-1-quinolin-5-yl-1H-pyrazole-4-carbonyl)-guanidine hemiethanolate of claim 6 which is characterized by an x-ray powder diffraction pattern further comprising peaks at about 8.55, 12.31, 14.11, 16.91, 23.44, 24.88 and 25.22, degrees two-theta.

8. A pharmaceutical composition comprising a compound selected from a compound from any one of claim 1-7 and a pharmaceutically acceptable vehicle, diluent or carrier.

9. A method for preparing N-(5-cyclopropyl-1-quinolin-5-yl-1H-pyrazole-4-carbonyl)-guanidine monoethanolate comprising:
forming a solution comprising N-(5-cyclopropyl-1-quinolin-5-yl-1H-pyrazole-4-carbonyl)-guanidine in ethanol at a concentration that is about the point of saturation of said compound in ethanol; and
crystallizing N-(5-cyclopropyl-l-quinolin-5-yl-1H-pyrazole-4-carbonyl)-guanidine monoethanolate from said solution.

10. A method of claim 9 wherein said crystallization is performed by cooling said solution to a temperature sufficient to effect such crystallization.

11. A method of claim 9 wherein said crystallization is performed by removal of ethanol from said solution by evaporation of an amount sufficient to effect such crystallization.

12. A method for preparing N-(5-cyclopropyl-1-quinolin-5-yl-1H-pyrazole-4-carbonyl)-guanidine hemiethanolate comprising:
forming a solution comprising N-(5-cyclopropyl-1-quinolin-5-yl-1H-pyrazole-4-carbonyl)-guanidine in ethanol at a concentration that is about the point of saturation of said compound in ethanol;
crystallizing N-(5-cyclopropyl-1-quinolin-5-yl-1H-pyrazole-4-carbonyl)-guanidine monoethanolate from said solution; and
subjecting the N-(5-cyclopropyl-1-quinolin-5-yl-1H-pyrazole-4-carbonyl)-guanidine monoethanolate to drying conditions to form N-(5-cyclopropyl-1-quinolin-5-yl-1H-pyrazole-4-carbonyl)-guanidine hemiethanolate.

13. A method of claim 12 wherein said drying conditions comprise a vacuum.

14. A method of claim 12 wherein said drying conditions comprise heat.

15. A method of claim 14 wherein said heat is at a temperature of about 40° C. to about 45° C.

16. A method of claim 12 wherein said drying conditions comprise heat and a vacuum.

17. A method of claim 16 wherein said heat is at a temperature of about 40° C. to about 45° C. and said vacuum is at or less than about 15 mm Hg.

18. A method of claim 12 wherein the N-(5-cyclopropyl-1-quinolin-5-yl-1H-pyrazole-4-carbonyl)-guanidine monoethanolate is subjected to drying conditions for at least about five hours.

19. A method of claim 17 wherein the N-(5-cyclopropyl-1-quinolin-5-yl-1H-pyrazole-4-carbonyl)-guanidine monoethanolate is subjected to drying conditions for at least about five hours.

20. A method of claim 12 wherein said crystallization is performed by cooling said solution to a temperature sufficient to effect such crystallization.

21. A method of claim 16 wherein said crystallization is performed by cooling said solution to a temperature sufficient to effect such crystallization.

22. A method of claim 12 wherein said crystallization is performed by removal of ethanol from said solution by evaporation in an amount sufficient to effect such crystallization.

23. A method of claim 16 wherein said crystallization is performed by removal of ethanol from said solution by evaporation in an amount sufficient to effect such crystallization.

24. A therapeutic method comprising administering to a mammal in need of preventing or reducing tissue damage resulting from ischemia or hypoxia a therapeutically effective amount of a compound selected from a compound of any one of claims 1-7 or a therapeutically effective amount of a pharmaceutical composition comprising said compound.

25. A method of claim 24 wherein said mammal is a human.

26. A method for preparing N-(5-cyclopropyl-1-quinolin-5-yl-1H-pyrazole-4-carbonyl)-guandine mesylate comprising combining a compound selected from N-(5-cyclopropyl-1-quinolin-5-yl-1H-pyrazole-4-carbonyl)-guanidine monoethanolate and N-(5-cyclopropyl-l-quinolin-5-yl-1H-pyrazole-4-carbonyl)-guanidine hemiethanolate, in an aprotic solvent, with methansulfonic acid at a temperature of about 40° C. to about 80° C.

27. A method of claim 26 wherein said aprotic solvent is tetrahydrofuran and said temperature is about 50° C. to about 60° C.

28. A method for preparing a pharmaceutical composition comprising preparing N-(5-cyclopropyl-1-quinolin-5-yl-1H-pyrazole-4-carbonyl)-guandine mesylate according to a method selected from a method of any one of claim 26 or 27 and combining said N-(5-cyclopropyl-1-quinolin-5-yl-1H-pyrazole-4-carbonyl)-guandine mesylate with a pharmaceutically acceptable vehicle, diluent or carrier.
